(19) United States
(12) Patent Application Publication
(10) Pub. No.: US 2009/0105752 A1
(43) Pub. Date: Apr. 23, 2009

(54) Hypodermic stiching needle

(76) Inventors: Harry Shonteff, San Francisco, CA (US); Freeman Grant Chambers, Irvine, CA (US)

Correspondence Address: Walt Froloff, 273D Searidge Rd, Aptos, CA, 95003, US

(21) Appl. No.: 11/975,868

(22) Filed: Oct. 22, 2007

(51) Int. Cl. A61B 17/06; A61B 17/04

(52) U.S. Cl. 606/223; 606/228

(57) ABSTRACT

The present invention discloses an apparatus for a hypodermic stitching needle, one in which the hypodermic hollow tube has an insert slot for a thread insertion, thread which extends through the inside hypodermic tubular channel to a beveled tip. The hollow needle channel protects the suture from tear and the needle tip provide increased thread control to the needle user.

### BACKGROUND

1. Field of the Invention

The present invention generally relates to stitching needles and more specifically, to very small hypodermic stitching needles for micro dimension precision.

Precision stitching needles in the last several decades has grown more widely in the medical industry because simply, surgeons sew where they can to remove abnormalities or install continuing smaller medical devices. It is a skill honed and mastered early, used virtually in every procedure they are called upon to perform. Originating from basic fabric sewing, it is not that difficult and with practice relatively safe. However, the trend in surgery of implanting medical devices has grown while device size has shrunk.

Some medical device manufacturing companies are now offering suturing systems and devices aimed at delivering the tactile control and precision associated with open procedures to minimally invasive surgery. As an added bonus, in the process of improving laparoscopic suturing, many manufacturers are also introducing safer technologies designed to protect the surgeon, or staff and the patient from the risks of needlestick injuries and infection. Thus better ways of reducing injury from needles through needle control are needed.

Present surgical procedures are challenged for device placement in difficult if not thought to be impossible locations previously and much smaller suturing work spaces. As medical devices grow smaller, components must become stronger to handle the resulting stresses, and surgical techniques and tools must change to meet the challenge of working with yet smaller devices with smaller working spaces. This applies to endoscopic as well as more traditional medical procedures.

Some procedures for device implantation have proven inadequate because weakened attachment dislodged devices. The securing of many medical devices requires suturing into tissue and onto new and tougher artificial materials, weaves and fabrics. The securing of these potentially dislodged devices is problematic, requiring smaller needles. Smaller needles generally means weaker needles, and any breakages from weakened sutures or overstressed needles adds to the operation risk.

Moreover, sewing with thin suture on difficult to sew small devices requires the use of a needle with a 0.0014 of a inch diameter or smaller. Furthermore, as part of the assembly process for many class 3 medical devices the standard needle sizes do not provide protection for the suture. Take for instance sewing stents to graft material as taught by Shonteff patent No. U.S. Pat. No. 6,295,940, discloses that the needle will come in contact with the feet of the sewing device, increasing the possibility the procedure may brake or damage the suture. Also when sewing on smaller devices such as coronary stents or other very small medical devices, needle contact with the stent will often tear the suture. Sewing on small tubular devices as introduced by Sew Fine™ LLC in 2002 with the small arm 301 lock stitch machine mitigates this problem and has been used to sew small tubular class 3 medical devices. However, the needle often touches the medical device that it is sewing. This is unacceptable in most cases for the obvious reasons. Where the risk is too high, a particular otherwise helpful medical procedure cannot be used. What is needed are smaller stitching devices, devices which can suture without tearing the tissue or the compromising the thread. What are needed are stitching devices which give the surgeon more precise thread control.

Very Small Tubular Devices

Often medical devices are made of very small tubular shapes to enable deployment in the artery. Many products like Endovascular stent grafts for abdominal and thoracic aortic aneurysms are made of tubular shaped graft material that is ether hand sewn together or precision sewn by machine like the small arm lock stitch machine as taught by Sew Fine™, in 2002 or on sewing devices like the Endovascular deployment machines used to sew deployment sheaths as taught by Sew Fine™, in 1997 and additional equipment provided in 2006. Medical devices used in procedures to support blood vessels, such as Endovascular stent graft, and devices to keep a vessel open, as in coronary stents, it is often the case that the devices are smaller then can be sewn mechanically because the precision cannot meet the dimensional requirements of the work. What is needed are devices which can be used in sewing stents to graft material smaller then 0.0850 of a inch. This is currently not possible because of the extremely small dimensions, restricting devices which can support the mechanical stresses, and work space available for suturing.

The evolving requirements of medical devices and other non-medical devices press the envelope for sewing on smaller parts and yet smaller parts. In attaching Endovascular devices, it is often the case that a sewn device requires a smaller more protective stitching needle then what is currently available. With new technologies evolving in the coronary and other endovascular devices, it is necessary to sew closer to a stent or device than is currently possible. What are needed as smaller stitching needle small enough to work around stent devices, yet strong enough not to break during the procedure with thread strong enough to last after the procedure.

Emerging medical industry extremely fine weaves and screens require sewing between the fibers without damaging the material. Currently, it is common to sew with microscopes or vision systems mounted to the sewing equipment, which provide a microscopic view of the sewing area. However, even this advantage does not support bending of the needle to conform to specific machine requirements. Moreover, the current needle technology does not offer protection for the thread and or suture as required for sewing small tubular devices to stents. Equally lacking is the capability for sewing of tissue to tissue or tissue to medical devices as a anchoring step, used in endovascular deployment or other surgical procedures.

What are needed are needle and stitching devices capable of sewing class 3 medical devices like Endovascular stent grafts, for the treatment of abdominal aortic aneurysms, thoracic aortic aneurysm coronary stent graft, and many other applications. Furthermore, heart valves and many other small hard to sew devices frequently brake thread or suture because the thread is overstressed in the procedure. What are needed are stronger micro sized needles using the small arm lock stitch as taught by Sew Fine™, and the sewing device by Shonteff, U.S. Pat. No. 6,295,940, which can take advantage of new and stronger surgical stitching needles in smaller and yet smaller medical devices.

### SUMMARY

The present invention discloses a system for an eyeless surgical stitching in hypodermic needle dimensions. The eyeless construction protects the suture during the suturing procedure. The invention comprises a tubular micro dimension hypodermic needle; a needle channel axially encased inside the needle; a needle shank coupled to support the needle; a small opening upstream of the needle distal end for thread insertion; and an oblique cross section opening at needle distal end. Thus a thread or suture is threaded through the upstream opening of the tubular needle, threading continuing through the needle inside channel and through the needle distal end tip opening, allowing the needle contact with sewing object without abrasive contact with the suture or thread.

The invention can have the thread input opening designed as a slot, oval, circular, rectangular, triangular. Other variations can include needle distal end opening of oblique cross section for various oblique to cross section angles. The tip can have a notch side opening for various thread or suture loop configurations and the needle tubular channel of can have cross sections of circular, oval, rectangular, triangular, or other areas and shapes.

### BRIEF DESCRIPTION OF DRAWINGS

Specific embodiments of the invention will be described in detail with reference to the following figures.

FIG. 1 is a front view illustration of a hypodermic stitching needle according to an embodiment of the present invention.

FIG. 2a is a perspective view illustration of a hypodermic stitching needle thread inlet and tip outlet according to an embodiment of the present invention.

FIG. 2b and FIG. 2c illustrate a notch of a hypodermic stitching needle tip according to an embodiment of the present invention

FIG. 3 is a front view illustration of a hypodermic stitching needle and stitching bobbin according to an embodiment of the present invention.

FIG. 4 is a front view illustration of a hypodermic stitching needle and hook type looper according to an embodiment of the present invention.

FIG. 5 is a front view illustration of a hypodermic stitching needle and a chain stitch looper according to an embodiment of the present invention.

FIG. 6 is a front view illustration of a hypodermic stitching needle and a hook for lockstitch according to an embodiment of the present invention.

FIG. 7 is a front view illustration of a hypodermic stitching needle and looper according to an embodiment of the present invention.

FIG. 8 is a front view illustration of a curved hypodermic stitching needle and according to an embodiment of the present invention.

FIG. 9 is a front view illustration of a curved hypodermic stitching needle and bobbin according to an embodiment of the present invention.

FIG. 10 is a front view illustration of a curved hypodermic stitching needle and hook type looper according to an embodiment of the present invention.

FIG. 11 is a front view illustration of a curved hypodermic stitching needle and chain stitch looper according to an embodiment of the present invention.

FIG. 12 is a front view illustration of a curved hypodermic stitching needle and hook for lockstitch according to an embodiment of the present invention.

FIG. 13 is a front view illustration of a curved hypodermic stitching needle and looper according to an embodiment of the present invention

### DETAILED DESCRIPTION

In the following detailed description of embodiments of the invention, numerous specific details are set forth in order to provide a more thorough understanding of the invention. However, it will be apparent to one of ordinary skill in the art that the invention may be practiced without these specific details. In that light, well-known features have not been described in detail to avoid unnecessarily complicating the description.

### Objects and Advantages

The present invention discloses a hypodermic surgical stitching needle for very small dimension applications. A hypodermic stitching needle is a hollow needle, not unlike a hypodermic needle used with a syringe, but without a syringe and used for a different purpose.

As with current hypodermic needles and their associated syringes, the hypodermic stitching needle can be designated for single use because they are difficult to decontaminate and need sharpening after repeat use. However, single use is not the case for other embodiments.

In an embodiment of the invention, hypodermic stitching needles are made from a stainless steel tube drawn through progressively smaller dies to make the needle. The end is beveled or oblique, to create a sharp pointed tip. This allows the needle to easily penetrate the stitching object as well as transfers threading control to the point where the stitching activity must be most precise. Although very small, the manufacturing of such hypodermic needles are known to one skilled in the art. In addition, plastic needle embodiments may be more flexible and not scratch the stent wire.

It an object of the present invention to provide embodiments designed to protect the suture or thread from abrasive or friction stress loading during the surgical procedures.

Another object of the invention is to adapt a micro-size surgical needle to many types of sewing devices.

Another object of the invention is to increase thread control by transferring the needle-to-thread coupling, ordinarily the eye away from the needle tip, directly to the needle tip.

Another object of the invention is to maximize the thread bend angles by needle design in order to minimize thread stresses.

Another object of the invention is to adapt the needle to wet suture use, as required in many surgical applications.

Another object of the invention is design of straight or curved as to accommodate the sewing device requirements.

Another object of the invention is sewing small tubular devices 0.0850 of a inch or smaller.

An object of the invention is to provide a sewing needle for adaptation to any commercially produced stitch including but not limited to stitch type 101, 102, 103, 105, 201, 202, 204, 205, 301, 302, 303, 304, 305, 306, 307, 308, 309, 310, 311, 312, 313, 314, 315, 316, 401, 402, 403, 404, 405, 406, and types 407, 501, 502, 503, 504, 505, 506, 507, 508, 509, 510, 511, 512, 513, 514, 515, 516, 517, 518, 519, 520, 521, 522, 601, 602, 603, 604, 605, 606, 607, as taught by the ASTM standards related to stitches and seams

### Embodiments of the Invention

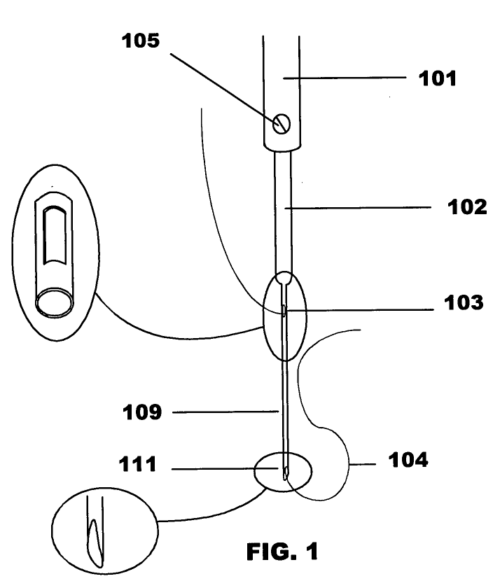

FIG. 1 is a front view illustration of a hypodermic stitching needle. The FIG. 1 embodiment illustrates an eyeless micro sized hypodermic surgical stitching needle. Instead of thread to a needle eye coupling, the suture 4 is instead threaded into a tubular channel 9 through the tubular channel thread inlet or thread slot 3 on the needle side opposite the needle distal end 11, thread 4 extending on through the needle tubular channel 9 and continuing out of the needle distal end opening 11. The tubular hypodermic needle 9 shield the suture in the needle channel 9 threading to the target sewing object without friction contact with the sewing object. The needle shank 2 is generally rigidly couples the needle 9 to the sewing mechanism by the needle clamp. In some embodiments, the shank 2 is larger in diameter than the portion that penetrates the object material. Many type of shank types can be used, include the round, single or flat slab, double or flat slab and notched. The FIG. 1, the needle shank 2 supports the needle 9 and is coupled to a needle bar 1 with a coupling or needle clamp fastener 5. The needle clamp fastener 5 or mounting screw is used to tighten the needle to the needle bar 1, the driving device in some embodiments of the invention. The needle distal end opening 11 or tip, has a bevel tip, ie an oblique cross section opening, creating a sharp pointed tip used for sewing object penetration and for thread protection. In other embodiments, the tip may also be a cutting point, ball point, wedge point and others. The thread portion in the hypodermic needle channel 9 is protected against friction contact with the sewing object. The needle tip protects against direct contact with the target sewing object as the beveled tip penetrates the sewing object carrying the thread into the sewing object behind the sharp tip.

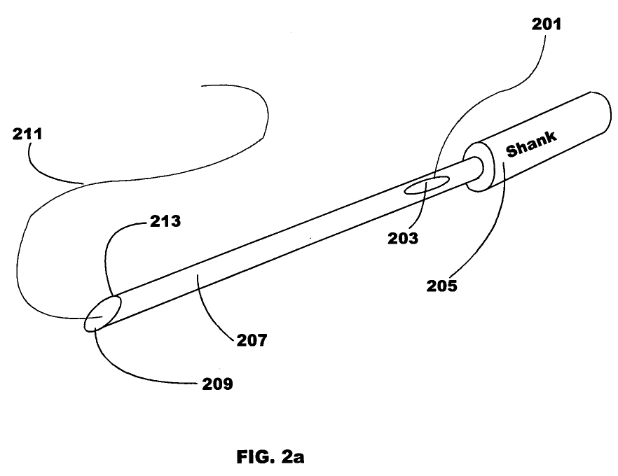

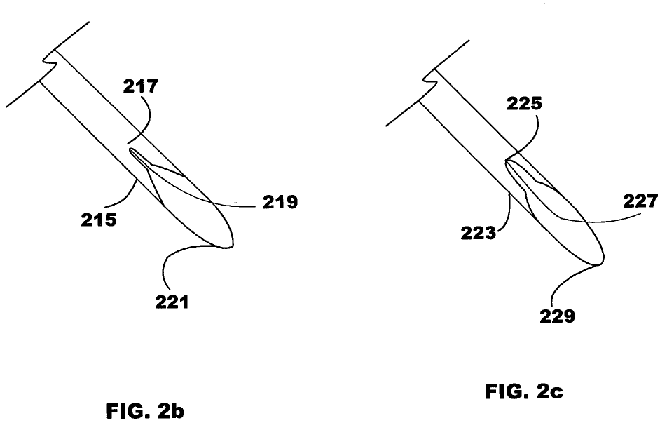

FIG. 2a is a perspective illustration of a hypodermic stitching needle tip, thread outlet, and needle shank side thread inlet.

The needle thread inlet orifice 203, channel 207 and outlet tip 209 collectively substitute for the “eye” of the needle. The suture 201 is threaded into the inlet orifice 203 and extended through the inside channel 207 hypodermic and out 211 through the tip outlet 209 which in some embodiments is a beveled tip, whose sharpness can vary on the needle material, target sewing object toughness and procedure requirements.

For some very small needle applications, 0.0023 inches and smaller, thread from Biogeneral™ PTE monofilament Type 74 size 0.0023 can be used. Alternatively, Surgical Specialties™ corp., Genzyne™ and W.L. Gore™ suture can be made of braided PTFE or non-braided PTFE also PET or any standard surgical monofilament.

Threading the needle can be problematic because of the very small dimensions of the thread inlet 203 and lack of visual understanding inside the needle channel 207 during the threading process. The needle can be threaded with the aid of a small threading guide wire or via a thread suction mechanism to aid coax the thread 201 through the tiny thread inlet 203, channel 207 and outlet tip 209. Also, in some very small embodiments of the invention, the needle threading will require a microscope for visual aid and mechanical coordination.

The hypodermic needle tube 207 or thread housing need not be cylindrical, The needle tube channel cross section can be circular, oval, rectangular, triangular, or almost any asymmetric polygon, constrained by costs, strength and material properties. The needle can be made of stainless steel or other rigid tubular material depending on the requirements of the sewn object and procedure to be followed. The tip 209 of the hypodermic needle can be oblique at different angle steepness to conform with alternate thread loop formations. In another embodiment, the tip will have a slot incorporated at the trailing tip edge to position the suture in the direction required by the thread catching sewing device, such as a bobbin or other stitch cooperating accoutrement such as a looper, spreader, or hook to catch the suture or thread. The hypodermic needle 207 may also be coated, plated, or treated with a Teflon chrome nickel of other treatments to reduce friction resistance on the suture or thread, or to enable a outer surface that will be contacting the object being sewn. This needle may be comprised of many types of materials, including plastic, steel, stainless steel 300 or 400 class, composites etc. The material must be rigid, but not brittle as it must be flexible yet strong.

An embodiment of the invention transfers thread control to the hypo stitching needle tip from what is typically the “eye” of the needle, somewhere downstream of the needle tip. Needle tip thread control and channel also enhances sewing of wet suture, as is often the case in Laparoscopic Surgery, Thoracoscopic Surgery, Gastrointestinal Endoscopy and other surgical procedures.

Wet suture is difficult to work with because it can become “heavier”, adhering to anything in contact, and difficult to loop and tie the suture in the prescribed stitching. The invention tubular needle prevents thread contact with tissue thus the thread from wetting, becoming sticky, “heavier” and more difficult to control. The invention translates into higher dexterity for the surgeon in controlling the thread in surrounding wet tissue, all while forming thread loops necessary for enabling the needle access to specific locations otherwise difficult to access.

FIG. 2b and FIG. 2c illustrate a notch of a hypodermic stitching needle tip 221 229 respectively. In another embodiment of the invention, a thread catch notch 217 225 or slot is made on the trailing edge of the needle 215 223 beveled tip 221 229 respectively. The notch 217 225 is used in constraining the other wise freely twisting suture 219 227 while positioning and manipulating the thread to form the desired loop or loops. Sutures 217 225 and or threads become twisted from torsion and bending stresses. These affect the suturing as the thread “jumps” around without manipulation but by releasing stresses in unpredictable directions, thus affecting mechanical uniformity resulting in a skipped stitches. A notch 217 225 or slot constrains the thread 219 227, even under internal stresses and in the direction intended for a proper loop to be formed. Notch shapes can vary from a flat tee notch 217 to a more rounded notch 225, depending on the thread 217 225 and needle 215 223 and bevel tip 221 229 dimensions.

Yet another challenge in sewing small and very small objects is wet suture. Wet tissue makes the thread sticky, “heavy” and unwieldy. A thread catch slot 217 225 at the needle 215 223 tip predictably stabilizes the motion, providing control of wet heavy suture.

The notch 217 225 at the needle 215 223 tip can also be cut by the same methods used in creating the needle thread inlet opening; Drilling, milling, laser cutting, and Electrical discharge machining (EDM).

EDM is a machining method primarily used for hard metals or those that would be impossible to machine with traditional techniques. There are two main types of EDM machines: Conventional EDM, also called Sinker EDM or Ram EDM, and Wire EDM. Both methods are applicable. A limitation is that EDM is used where materials are electrically conductive. EDM can cut very small and or odd-shaped angles, intricate contours or cavities in pre-hardened steel without the need for heat treatment to soften and re-harden them as well as exotic metals such as titanium, hastelloy, kovar, inconel and carbide. Thus EDM is well suited to form even very small notches and thread inlet openings for many metal materials comprising these embodiments.

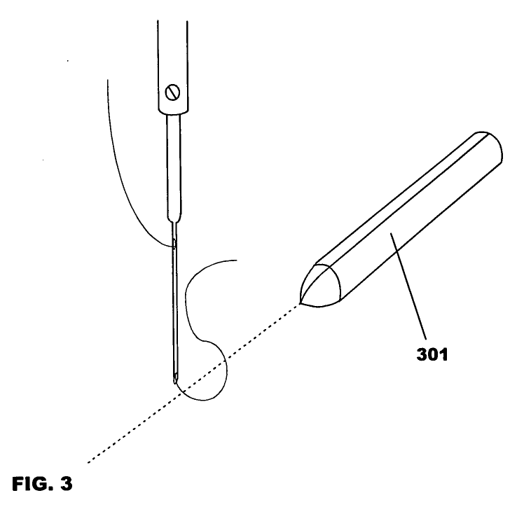

Many thread-receiving accoutrements for the stitching needle have been developed. Embodiments of the invention adopt these for application with a hypodermic stitching needle. FIG. 3 is a front view illustration of a hypodermic stitching needle and stitching bobbin 301.

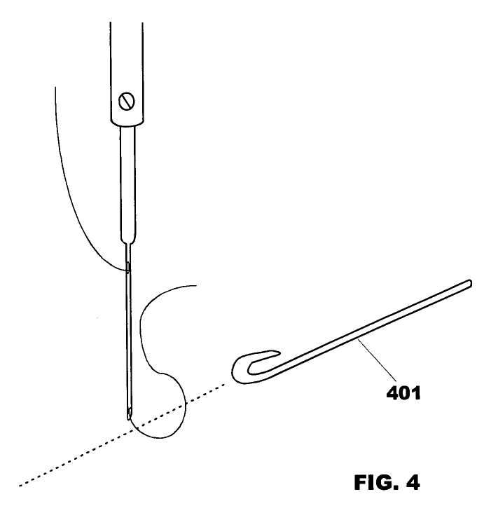

FIG. 4 is an illustration of a hypodermic stitching needle and hook type looper 401.

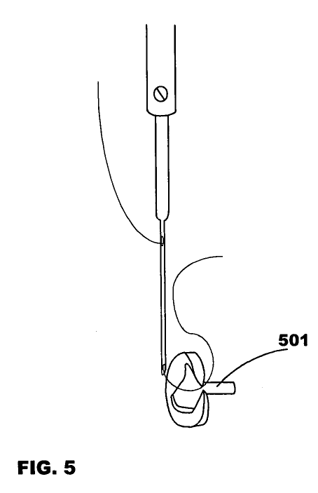

FIG. 5 is an illustration of a hypodermic stitching needle in application with a chain stitch looper 501.

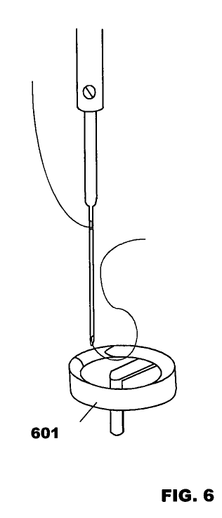

FIG. 6 is an illustration of a hypodermic stitching needle and a hook for lockstitch 601.

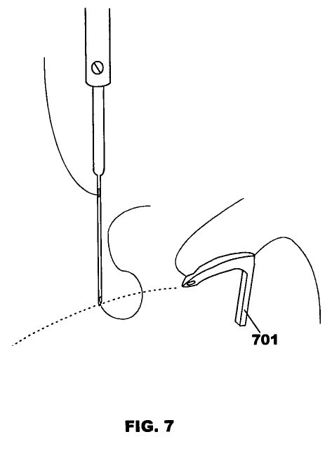

FIG. 7 is an illustration of a hypodermic stitching needle and a second thread looper 701.

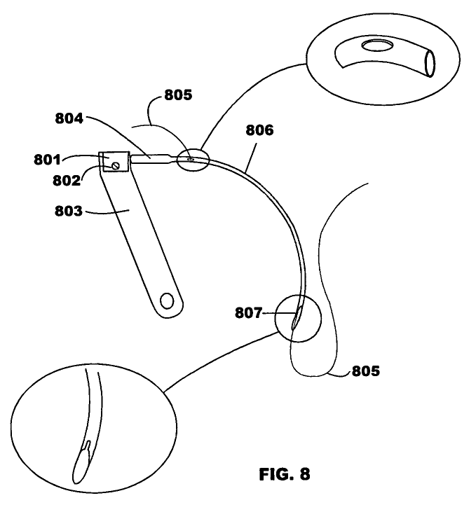

FIG. 8 is an illustration of a curved hypodermic stitching needle. In accordance with another embodiment of the invention, a needle clamp 801 couples the shank 804 to the needle bar 803 via a needle clamp mounting screw 802. The needle clamp is common to sewing mechanisms as a means of attaching the needle 806 to driver mechanism. The clamp mounting screw 803 is used to tighten the needle clamp 801 to the needle bar 803, which transfers the driving force or rotation from the power mechanism. The needle bar also guides the needle 806, straight or curved, to the sewing object or target.

The needle shank 804 couples the driving force from the power mechanism to the needle 806. Some embodiments of the invention have shank diameter dimensions larger and tapered to the needle 806. Other embodiments have shanks 804 which are round, single or flat slab, double or flat slab and notched.

The thread or suture 805 is introduced through the needle 806 shank 804 end inlet slot 808 and guided through the needle 806 inside channel to the needle tip 807 outlet. The needle tip 807, commonly referred to as the point maybe beveled at various steepness for various sharpness characteristics, or the tip maybe of rounded, ball point, wedge point or other type.

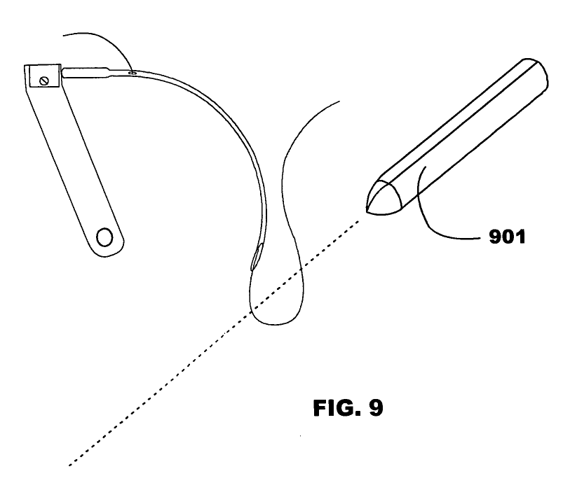

As embodiments of the invention, several thread receiving accoutrements for the curved stitching needle have been developed. Embodiments of the invention adopt these for application with a hypodermic stitching needle. FIG. 9 is an illustration of a curved hypodermic stitching needle and stitching bobbin 901.

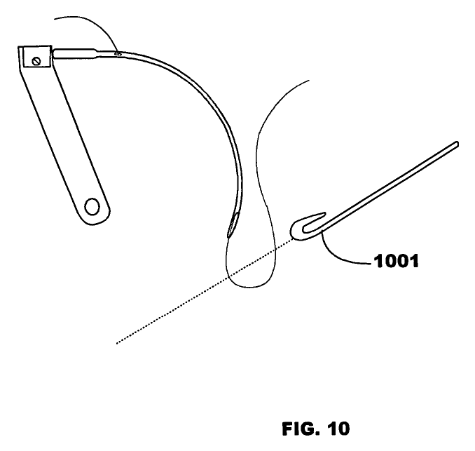

FIG. 10 is an illustration of a curved hypodermic stitching needle and hook type looper 1001.

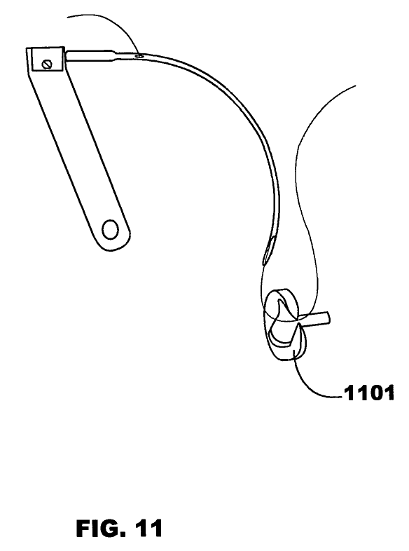

FIG. 11 is an illustration of a curved hypodermic stitching needle and chain stitch looper 1101.

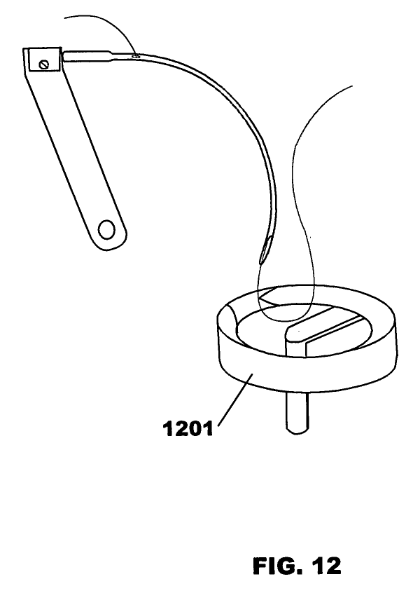

FIG. 12 is an illustration of a curved hypodermic stitching needle and hook for lockstitch.

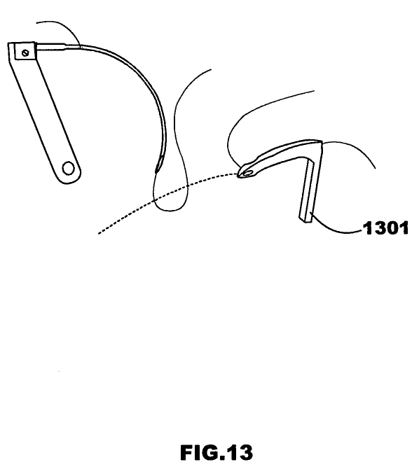

FIG. 13 is a an illustration of a curved hypodermic stitching needle and looper 1301 according to an embodiment of the present invention

Therefore, while the invention has been described with respect to a limited number of embodiments, those skilled in the art, having benefit of this invention, will appreciate that other embodiments can be devised which do not depart from the scope of the invention as disclosed herein. Other aspects of the invention will be apparent from the following description and the appended claims.

What is claimed is:

1. An eyeless hypodermic stitching needle comprising:
a tubular hypodermic needle wherein the hollow needle inside channel shields the suture in transport to the target sewing object;
a needle shank coupling a driver mechanism to the needle;
a thread inlet for suture insertion into the needle channel, at the needle shank end, and and with thread outlet at needle distal end,
whereby the suture is threaded through the needle shank end inlet, extending on through the needle channel and continuing out of the needle distal end outlet, transferring thread control to the needle tip for suturing, while protecting the suture from damage.

2. The hypodermic stitching needle in claim 1 further comprising the needle channel inlet as a slot, oval, circular, rectangular, triangular or angled outside to inside needle thickness.

3. The hypodermic stitching needle in claim 1 further comprising the needle distal end outlet of bevel or oblique cross section at various needle axis to tube cross section angles.

4. The hypodermic stitching needle in claim 1 further comprising at least one notch slot at the needle distal end bevel trailing edge for thread control and suture protection.

5. The hypodermic stitching needle in claim 1 wherein the needle inside channel cross section may be circular, oval, rectangular, triangular, or asymmetric polygon.

6. The hypodermic stitching needle in claim 1 wherein the shank is designed to fit the dimensional requirements of alternate sewing mechanisms.

7. The hypodermic stitching needle in claim 1 further comprising a needle distal end tip designed to assist in sewing of wet suture, shielding the suture from target moisture contact.

8. The hypodermic stitching needle in claim 1 wherein the stitching needle is straight or curved for accommodating various sewing application requirements or alternate thread control characteristics.

9. The hypodermic stitching needle in claim 1 further comprising a rigid needle of various needle material flexibility for application in various micro dimensional work spaces.

10. The hypodermic stitching needle in claim 1 further comprising suture material from a set of sutures comprising thread from Biogeneral PTE monofilament Type, Surgical Specialties corp., Genzyne and W.L. Gore suture braided PTFE or non-braided PTFE, PET or any standard surgical monofilament.

11. The hypodermic stitching needle in claim 1 wherein the needle may be composed of tough flexible types of materials, including plastic, steel, stainless steel 300 or 400 class, and composites
